(19) United States
(12) Patent Application Publication
(10) Pub. No.: US 2024/0138442 A1
(43) Pub. Date: May 2, 2024

(54) ORAL FELINE FEED AND METHODS FOR CONTROLLING FLEA INFESTATIONS IN A FELINE

(71) Applicant: In the Bowl Animal Health, Inc., Nashville, TN (US)

(72) Inventors: Joseph R. Winkle, Carmel, IN (US); Robin S. Readnour, Nashville, TN (US); Kevin E. Willard, Knightstown, IN (US)

(21) Appl. No.: 18/543,411

(22) Filed: Dec. 18, 2023

Related U.S. Application Data
(63) Continuation of application No. PCT/US22/34685, filed on Jun. 23, 2022.
(60) Provisional application No. 63/214,967, filed on Jun. 25, 2021.

(51) Int. Cl. A23K 20/163; A23K 50/48; A61K 9/00; A61K 31/7048; A61K 45/06; A61P 33/14

(52) U.S. Cl. CPC A23K 20/163; A23K 50/48; A61K 9/0056; A61K 31/7048; A61K 45/06; A61P 33/14

(57) ABSTRACT

An oral feline feed and a method of controlling fleas in a feline in need thereof by orally administering to the feline a daily feed comprising an effective amount of a spinosyn for an effective time to thereby cause the amount of spinosyn in the feline's blood to rise to and be maintained at a therapeutically effective level for controlling fleas.

### RELATED APPLICATIONS

This application is a continuation of International Application Serial No. PCT/US2022/034685, filed Jun. 23, 2022, which claims priority to U.S. Patent Application Ser. No. 63/214,967, filed Jun. 25, 2021, the entire disclosures of both of which are hereby incorporated herein by reference.

### TECHNICAL FIELD

The teachings of this disclosure generally relate to a spinosyn, a feline feed that includes the spinosyn and a method of administering the spinosyn in a feed to control flea infestations in felines.

### BACKGROUND AND SUMMARY

Fleas are the most common of all external feline parasites, the most common of which is the cat flea, or Ctenocephalides felis. A flea infestation is usually terribly uncomfortable for most cats and can be the source of deadly disease. Flea bites can cause itching, and the consequent scratching by the cat may cause skin wounds that are vulnerable to infection. Further, the fleas themselves often carry infectious agents. For example, the cat flea can carry the larval stage of the tapeworm Dipylidium caninum and cats can become infected by ingesting fleas during self-grooming.

Further, fleas are instrumental in the transmission of “cat scratch disease” to humans. This is an infection with the bacterium Bartonella hensellae and is spread when fleas feed on blood. Cat scratch disease causes symptoms in humans that are unpleasant and can include, among other things, bumps or blisters at the scratch site, swollen lymph nodes, fatigue, headache, fever and body aches. One of the recommended ways to control human risk from fleas is to control the risk of infestation in cats.

Treatments currently available for controlling flea infestations in felines achieve varying degrees of success. Many treatments involve chemicals applied to indoor and outdoor surfaces, as well as to the feline. The chemicals used include a variety of carbamates, pyrethrins and pyrethroids, isoxazolines, certain macrocyclic lactones, insect growth regulators (including chitin synthesis inhibitors, juvenile hormone analogs, and juvenile hormones), nitromethylenes, neonicotinoids, pyridines and pyrazoles or fiproles. These compounds often have toxic side effects that are a problem for both the feline and its owner. In addition, there is evidence that the use of these chemicals may be ineffective due to insecticide resistance and treatment deficiencies. [M. K. Rust, The Biology and Ecology of Cat Fleas and Advancements in Their Pest Management: A Review, Insects 2017, 8 118.].

Topical treatments are a well-known method for controlling flea infestations in felines. While there are numerous ways to deliver these therapeutic agents to the haircoats and skins of felines, many of these methods are either ineffective and/or present safety risks to the feline or user during or after the dispensing activity. More particularly, because a physical connection at the skin level must be achieved between the applicator tip of the drug delivery device when the applicator tip is installed thereon, there is inherently a risk that the connection will be inadequate, thereby permitting some of the therapeutic agent to leak out of the device and into physical contact with the user. For example, in the case of uncooperative felines, it may be difficult to maneuver the dispenser with one hand and maintain the feline in place with the other hand, resulting in some, if not all, of the substance being spilled on the floor or on the person applying it instead of reaching the feline's skin. Not only is this leakage wasteful and messy, it also places the user at a heightened risk of suffering from a skin irritation or other such health concern, particularly if the user comes into direct contact with the agent.

Oral treatments are also available. However, to be effective, the feline owner must administer a treatment once every 30-90 days, for example. The extended time between treatments creates compliance issues when owners forget to administer doses.

Despite the availability of effective treatments, a recent study by The Harris Poll found that 33% of pet owners do not routinely protect their pets against fleas at all. Another study found that pet owners purchased, on average, only 4 months of flea prevention products per year per pet, despite being told that pets needed to be given flea prevention treatments year-round. Thus, there continues to be a need for relatively safe, effective agents for controlling flea infestations on felines that are easier for owners to remember to use.

Surprisingly, it has been discovered by the inventors that spinosyns, such as spinosad, can provide improved control over flea infestations in felines when orally administered in smaller, more frequent/chronic doses. The administration is discussed below as being combined with feed. However, it is also contemplated that the spinosyns may be administered by itself or in a dosage form other than feed, such as a chew, tablet, liquid, gel or other suitable form for oral administration. Advantageously, by using smaller, more frequent doses, less total active material is required over the same time period to control flea infestations. Assume for example, that 51 mg of spinosyn/kg of feline body weight is needed for a single dose in a 30-day (1-month) period according to the prior art approach to reach and maintain a therapeutically effective concentration of spinosyn in the feline's blood for continued flea control. With the inventive approach of smaller and more frequent doses, as little as 0.21-1.4 mg of spinosyn/kg of feline body weight, or 6.375-42 mg of spinosyn/kg of feline body weight cumulative over the same 30-day period.

Advantageously, the total amount of spinosyn required for a therapeutically effective once-monthly dose can be reduced by 10-87.5% by converting to daily administration. However, from a practical perspective, at least two problems arise: (1) creating a homogenous feed; and (2) analytical control testing for a very small dose of spinosyn may be difficult to accomplish. The analytical matrix from feeds can be quite complex and difficult to assay. Assays will be in the parts per million to billion range for some needed dose and feed concentrations. Thus, it is possible that one of skill in the art may opt to increase the daily dose such that the total of the daily doses over the course of one month equals the prior art once-monthly dose or is even higher, for example, 200% of the prior art once-monthly dose. This may be done to help ensure homogeneity as well as increase assay accuracy and decrease analytical variability when administering the dose as part of a feed.

The method and composition taught herein have the further advantage of encouraging compliance because the smaller doses of a spinosyn can be incorporated into a feed. Since owners naturally follow a daily feeding regimen in any event, this makes it less likely that owners will forget or neglect to administer the spinosyn. Thus, this disclosure provides a method for prolonged control of fleas in a safer and more effective manner than that achieved with previously known treatment methodologies. All the owner need remember is to feed their pet as they normally would.

Spinosyns are naturally derived fermentation products. They are macrolides produced by cultivation of Saccharopolyspora spinosa. The fermentation of S. spinosa produces many factors, including spinosyn A and spinosyn D (also called A83543A and A8354D). Spinosyn A and spinosyn D are the two spinosyns that are most active as insecticides. A product comprised mainly of these two spinosyns is available commercially under the generic name “spinosad.” The major spinosyn factor, spinosyn A, is particularly known to have an excellent human and feline safety and toxicological profile.

Each spinosyn has a 12-membered macrocyclic ring that is part of an unusual tetracyclic ring system to which two different sugars are attached, the amino-sugar forosamine and the neutral sugar 2N,3N,4N-(tri-O-methyl)rhamnose. This unique structure sets the spinosyns apart from other macrocyclic compounds.

Spinosyn A was the first spinosyn isolated and identified from the fermentation broth of S. spinosa. Subsequent examination of the fermentation broth revealed that S. spinosa produced a number of spinosyns that have been called spinosyns A to J (A83543A to J). The primary components are spinosyns A and D. Additional spinosyns, lettered from K to W, have been identified from mutant strains of S. spinosa. The various spinosyns are characterized by differences in the substitution patterns on the amino group of the forosamine, at selected sites on the tetracyclic ring system and on the 2N,3N,4N-(tri-O-methyl)rhamnose group.

Boeck et al. described spinosyns A-H and J (which they called A83543 factors A, B, C, D, E, F, G, H and J), and salts thereof, in U.S. Pat. No. 5,362,634 (issued Nov. 8, 1994); U.S. Pat. No. 5,496,932 (issued Mar. 5, 1996); and U.S. Pat. No. 5,571,901 (issued Nov. 5, 1996). Mynderse et al. described spinosyns L-N (which they called A83543 factors L, M and N), their N-demethyl derivatives, and salts thereof, in U.S. Pat. No. 5,202,242 (issued Apr. 13, 1993); and Turner et al. described spinosyns Q-T (which they called A83543 factors Q, R, S and T), their N-demethyl derivatives, and salts thereof, in U.S. Pat. No. 5,591,606 (issued Jan. 7, 1997) and U.S. Pat. No. 5,631,155 (issued May 29, 1997). Spinosyns K, O, P, U, V, W and Y are described, for example, by Carl V. DeAmicis, James E. Dripps, Chris J. Hatton and Laura I. Karr in American Chemical Society's Symposium Series: Phytochemicals for Pest Control, Chapter 11, “Physical and Biological Properties of Spinosyns: Novel Macrolide Pest-Control Agents from Fermentation,” pages 146-154 (1997).

The spinosyns can react to form salts that are also useful in the methods and formulations of this disclosure. The salts are prepared using standard procedures for salt preparation. For example, spinosyn A can be neutralized with an appropriate acid to form an acid addition salt. The acid addition salts of spinosyns are particularly useful. Representative suitable acid addition salts include salts formed by reaction with either an organic or inorganic acid such as, for example, sulfuric, hydrochloric, phosphoric, acetic, succinic, citric, lactic, maleic, fumaric, cholic, pamoic, mucic, glutamic, camphoric, glutaric, glycolic, phthalic, tartaric, formic, lauric, stearic, salicylic, methanesulfonic, benzenesulfonic, sorbic, picric, benzoic, cinnamic and like acids.

The term “spinosyn” as used herein refers to an individual spinosyn factor (spinosyn A, B, C, D, E, F, G, H, J, K, L, M, N, O, P, Q, R, S, T, U, V, W or Y), an N-demethyl derivative of an individual spinosyn factor, a chemically modified spinosyn such as spinetoram, a salt of any of the aforementioned, a metabolite of any of the aforementioned, a physiologically acceptable derivative thereof, or a combination thereof.

Spinosyns also provide advantages because they are very effective against fleas with post-treatment residual protection, when the dosages described herein are used. Furthermore, spinosyns have no insecticidal cross-resistance to existing compounds. Thus, they are especially useful against flea populations on felines that have existing levels of resistance to currently used products. Spinosyns, therefore, can be used in integrated pest management (IPM) programs to extend the lifeline of commonly used products where resistance is not well developed or has not yet developed.

Systemic efficacy (e.g., ingestion of blood containing spinosyns by fleas) provides a different mode of exposure compared to topically applied formulations where contact with the flea at the skin surface is the mode of exposure. The advantages of oral systemic treatments and killing of fleas from their ingestion of blood, compared to topical applications and contact killing, include:
a) reduced exposure to the human applicator and children and objects in the feline's environment (e.g., flooring, carpets, furniture);
b) no worry about loss from exposure of the feline to water (lakes, streams, bathing, etc.) or from loss due to rubbing;
c) no concern about UV exposure and degradation;
d) no problems with oxidation from oils on skin, etc.; and
e) assurance that the entire dose is administered (compared to a topical application where some of the dose may drip off, nib off and/or remain in the dispensing tube immediately after treatment).

The formulations, or feeds, and methods of this disclosure may further include, in combination with the spinosyn, one or more other active substances having therapeutic efficacy. Such active substances include agents efficacious against fleas. Active substances may include, for example, isoxazolines, certain macrocyclic lactones, insect growth regulators (including chitin synthesis inhibitors, juvenile hormone analogs, and juvenile hormones), nitromethylenes, neonicotinoids, pyridines and pyrazoles.

The methods of this disclosure are carried out by administering the spinosyn to the feline in small, frequent doses. To facilitate routine dosing, the spinosyn administration may be carried out using a feed or chew. A number of different feeds are envisioned, provided the manufacturing process(es) and feed compositions do not have deleterious effects related to chemical stability, efficacy and safety on the spinosyn and, if applicable, other active substances. For example, feeds in the broad categories of dry, semi-moist, canned-retorted feeds, a treat, chews, a snack or other supplemental feed, or fresh refrigerated feeds may be adapted for use with this disclosure. The feline receives a maintenance quantity of spinosyn by consuming the feed or chew product on a weekly, semi-weekly or daily basis.

By incorporating smaller doses of spinosyn into an animal feed composition and administering it at an effective rate (most preferably daily), the blood level of the spinosyn rises over time until it reaches an optimal steady state where it can be maintained by a daily or substantially daily dosage. By contrast, when a spinosyn is orally administered in larger doses at lower frequency, e.g., a single treatment of a large dose that is administered via “treat” once in a 30-day period, the level of the spinosyn in the blood spikes at the time of the dose and then declines until the next dose is administered. The administration of a large dose at low frequency means that the feline must consume more spinosyn in each dose so that the blood level of the spinosyn does not fall below the necessary level for effective protection before the next dose.

Embodiment 1: A method of controlling a flea infestation in a feline in need thereof, comprising orally administering to said feline an effective amount of a spinosyn for an effective time at a frequency of at least 4 times per month.

Embodiment 2: The method of embodiment 1, wherein said feline is a domestic cat.

Embodiment 3: The method of any of embodiment 1 or 2, wherein said spinosyn is spinosad.

Embodiment 4: The method of any of embodiments 1-3, wherein said spinosyn is provided in a feed selected from the group consisting of dry cat food and wet cat food.

Embodiment 5: The method of any of embodiments 1-3, wherein said spinosyn is present in an amount of between about 7.5 mg/kg to 2400 mg/kg of a feed.

Embodiment 6: The method of any of embodiments 1-5, wherein said spinosyn is administered to said feline in an amount of between about 0.18 mg/kg and 17 mg/kg of body weight of said feline.

Embodiment 7: The method of any of embodiments 1-6, wherein the oral administration includes a feeding frequency selected from the group consisting of: at least 3 times per week, substantially daily and daily.

Embodiment 8: The method of any of embodiments 1-7, wherein said effective time is selected from the group consisting of at least one week and at least two weeks.

Embodiment 9: The method of any of embodiments 1-8, wherein said administration provides a therapeutically effective level of spinosyn in said feline's blood within a time period selected from the group consisting of within one week of the first administration of said spinosyn, and within two days of the first administration of said spinosyn.

Embodiment 10: The method of any of embodiments 1-9, wherein said administration provides a therapeutically effective level of spinosyn in said feline's blood for a period of time selected from the group consisting of: at least 45 days, at least 60 days, at least 90 days, at least 180 days and at least 365 days.

Embodiment 11: The method of any of embodiments 1-10, wherein said administration provides a concentration of spinosyn of between 7.5 ng/mL and 1020 ng/mL in said feline's blood for a time period selected from the group consisting of at least 30 days and at least 365 days.

Embodiment 12: The method of any of embodiments 1-11, wherein said spinosyn is administered for a number of days out of 30 days selected from the group consisting of at least 15 days and at least 20 days.

Embodiment 13: The method of any of embodiments 1-12, wherein said spinosyn is a component of a feed that comprises one or more other active substances.

Embodiment 14: The method of any of embodiments 1-13, further comprising discontinuing the administration of spinosyn for a number of days selected from the group consisting of at least 3 days and at least 7 days, wherein the feline's blood concentration of spinosyn is maintained at a therapeutically effective level.

Embodiment 15: The method of embodiment 14, further comprising resuming the administration of spinosyn after the discontinuing of the administration of spinosyn and thereby maintaining the feline's blood concentration of spinosyn at the therapeutically effective level.

Embodiment 16: The method of embodiment 1, wherein the spinosyn is a component of a chew.

Embodiment 17: The method of embodiment 16, wherein the oral administration includes a feeding frequency selected from the group consisting of: at least 3 times per week, substantially daily and daily.

Embodiment 18: A spinosyn for use in controlling fleas on a feline in need thereof, said spinosyn being administered in an effective amount to said feline for an effective time at a frequency of at least four times per month.

Embodiment 19: The spinosyn of embodiment 18, wherein said feline is a domestic cat.

Embodiment 20: The spinosyn of any of embodiment 18-19, wherein said spinosyn is spinosad.

Embodiment 21: The spinosyn of any of embodiments 18-20, wherein said spinosyn is provided in a feed selected from the group consisting of dry cat food and wet cat food.

Embodiment 22: The spinosyn of any of embodiments 18-21, wherein said spinosyn is present in an amount of between about 7.5 mg/kg to 2400 mg/kg of a feed.

Embodiment 23: The spinosyn of any of embodiments 18-22, wherein said spinosyn is administered to said feline in an amount of between about 0.18 mg/kg and 17 mg/kg of body weight of said feline.

Embodiment 24: The spinosyn of any of embodiments 18-23, wherein said administration includes a feeding frequency selected from the group consisting of: at least 3 times per week, substantially daily and daily.

Embodiment 25: The spinosyn of any of embodiments 18-24, wherein said effective time is selected from the group consisting of at least one week and at least two weeks.

Embodiment 26: The spinosyn of any of embodiments 18-25, wherein said administration provides a therapeutically effective level of spinosyn in said feline's blood within a time period selected from the group consisting of within one week of the first administration of said spinosyn, and within two days of the first administration of said spinosyn.

Embodiment 27: The spinosyn of any of embodiments 18-26, wherein said administration provides a therapeutically effective level of spinosyn in said feline's blood for a period of time selected from the group consisting of: at least 45 days, at least 60 days, at least 90 days, at least 180 days and at least 365 days.

Embodiment 28: The spinosyn of any of embodiments 18-27, wherein said administration provides a concentration of spinosyn of between 7.5 ng/mL and 1020 ng/mL in said feline's blood for a period of time selected from the group consisting of at least 30 days and at least 365 days.

Embodiment 29: The spinosyn of any of embodiments 18-28, wherein said spinosyn is administered for a number of days out of 30 days selected from the group consisting of at least 15 days and at least 20 days.

Embodiment 30: The spinosyn of any of embodiments 18-29, wherein the spinosyn is a component of a feed that comprises one or more other active substances.

Embodiment 31: The spinosyn of any of embodiments 18-30, further comprising discontinuing the administration of spinosyn for a number of days selected from the group consisting of at least 3 days and at least 7 days, wherein the feline's blood concentration of spinosyn is maintained at a therapeutically effective level.

Embodiment 32: The spinosyn of embodiment 31, further comprising resuming the administration of spinosyn after the discontinuing of the administration of spinosyn and thereby maintaining the feline's blood concentration of spinosyn at the therapeutically effective level.

Embodiment 33: The spinosyn of embodiments 18-32, wherein the spinosyn is a component of a chew.

Embodiment 34: The spinosyn of embodiment 33, wherein the administration includes a feeding frequency selected from the group consisting of: at least 3 times per week, substantially daily and daily.

Embodiment 35: A feed or chew for controlling fleas in a feline, said feed or chew comprising a therapeutically effective amount of a spinosyn to control a flea infestation when administered to said feline for an effective time at a frequency of at least four times per month.

Embodiment 36: The feed or chew of embodiment 35, wherein said feline is a domestic cat.

Embodiment 37: The feed or chew of any of embodiment 35 or 36, wherein said spinosyn is spinosad.

Embodiment 38: The feed or chew of any of embodiments 35-37, wherein said spinosyn is provided in a feed selected from the group consisting of dry cat food and wet cat food.

Embodiment 39: The feed or chew of any of embodiments 35-38, wherein said spinosyn is present in an amount of between about 7.5 mg/kg to 2400 mg/kg of a feed.

Embodiment 40: The feed or chew of any of embodiments 35-39, wherein said spinosyn is administered to said feline in an amount of between about 0.18 mg/kg and 17 mg/kg of body weight of said feline.

Embodiment 41: The feed or chew of any of embodiments 35-40, wherein said administration includes a feeding frequency selected from the group consisting of: at least 3 times per week, substantially daily and daily.

Embodiment 42: The feed or chew of any of embodiments 35-41, wherein said effective time comprises administering the feed or chew for a period of time selected from the group consisting of at least one week and at least two weeks.

Embodiment 43: The feed or chew of any of embodiments 35-42, wherein said administration provides a therapeutically effective level of spinosyn in said feline's blood within a period of time selected from the group consisting of: within one week of the first administration of said feed or chew and within two days of the first administration of said feed or chew.

Embodiment 44: The feed or chew of any of embodiments 35-43, wherein said administration provides a therapeutically effective level of spinosyn in said feline's blood for a period of time selected from the group consisting of: at least 45 days, at least 60 days, at least 90 days, at least 180 days and at least 365 days.

Embodiment 45: The feed or chew of any of embodiments 35-44, wherein said administration provides a concentration of spinosyn of between 7.5 ng/mL and 1020 ng/mL in said feline's blood for a period of time selected from the group consisting of at least 30 days and at least 365 days.

Embodiment 46: The feed or chew of any of embodiments 35-45, wherein said feed or chew is administered at a frequency selected from the group consisting of: at least 15 out of 30 days, and at least 20 out of 30 days.

Embodiment 47: The feed or chew of any of embodiments 35-46, wherein said feed or chew comprises one or more other active substances.

Embodiment 48: The feed or chew of any of embodiments 35-47, further comprising discontinuing the administration of the feed or chew for a number of days selected from the group consisting of at least 3 days and at least 7 days, wherein the feline's blood concentration of spinosyn is maintained at a therapeutically effective level.

Embodiment 49: The feed or chew of embodiment 48, further comprising resuming the administration of the feed or chew after the discontinuing of the administration of the feed or chew and thereby maintaining the feline's blood concentration of spinosyn at the therapeutically effective level.

In an aspect of any of the embodiments, administration for controlling a flea infestation maintains a concentration of spinosyn of at least 7.5 ng/ml and not more than 1020 ng/ml in said feline's blood for at least 30 days. More preferably, administration maintains a concentration of spinosyn of at least 7.5 ng/ml and not more than 510 ng/ml in said feline's blood for at least 30 days. More preferably, administration maintains a concentration of spinosyn of at least 15 ng/ml and not more than 383 ng/ml in said feline's blood for at least 30 days. Still more preferably, administration maintains a concentration of spinosyn of at least 37.5 ng/ml and not more than 340 ng/ml in said feline's blood for at least 30 days.

In an aspect of any of the embodiments, administration for controlling a flea infestation maintains a concentration of spinosyn of at least 7.5 ng/ml and not more than 1020 ng/ml in said feline's blood for at least 365 days. More preferably, administration maintains a concentration of spinosyn of at least 7.5 ng/ml and not more than 510 ng/ml in said feline's blood for at least 365 days. More preferably, administration maintains a concentration of spinosyn of at least 15 ng/ml and not more than 383 ng/ml in said feline's blood for at least 365 days. Still more preferably, administration maintains a concentration of spinosyn of at least 37.5 ng/ml and not more than 340 ng/ml in said feline's blood for at least 365 days.

### BRIEF DESCRIPTION OF THE FIGURES

FIG. 1. A graph of the arithmetic mean efficacy for treatment Group 2 (stars) and Group 3 (left hash). Percent efficacy=[AM fleas CONTROL−AM fleas TREATED]/AM fleas CONTROL Efficacy measured at 2, 7, 14, 21, 30, and 37 days after Initial Treatment.

### DESCRIPTION

All ratios, percentages, and parts discussed herein are “by weight” unless otherwise specified.

The term “controlling a flea infestation” refers to preventing, treating, minimizing or eliminating an infestation by fleas on a feline.

The term “flea” refers to any member of the order Siphonaptera. The term “flea” includes the egg, larval, pupal, and adult stages of development.

The term “feline” refers to any member of the subfamily Felidae, which includes such species as the domestic cat, bobcats, wildcats, ocelots, members of the genus Lynx, Pallas's cat and cougars.

In carrying out the methods of this disclosure, a “feed” is an animal feed or treat, snack or other supplemental feed that may be administered daily or substantially daily. By using different forms of feed, e.g., kibble and treats, a pet owner may vary the feline's meals and snacks from time to time while still conveniently administering a daily dose of spinosyn.

The term “chew” refers to a treat that has flavor and aromatic properties that are appealing to a feline, but typically has no nutritional value. In carrying out the methods of this disclosure, a “feed” and/or a “chew” may be used interchangeably.

The term “effective time,” also referred to herein as “effective duration,” for the purposes of this disclosure includes at least the duration of feed administration needed to bring the level of spinosyn in the feline's blood to a sufficiently high level for controlling fleas, i.e., a “therapeutically effective” level. In some embodiments, the effective time may be as little as three days. In other instances, the effective time may be seven days or fifteen days or longer. As discussed below, the effective time will vary based on how frequently the feed or spinosyn is administered.

As just alluded, the “effective time” will vary as a function of the frequency at which the feed is administered. The term “effective frequency” as used herein means the number of feedings over a given time that produce a therapeutically effective concentration of spinosyn in the feline's blood. In all events, the term “effective frequency” as used herein contemplates multiple feedings including the spinosyn per month. One of skill in the art will appreciate that the spinosyn may be administered in a range of frequencies. For example, the spinosyn may be administered at a frequency of daily, every other day, every third day, once per week or even at inconsistent time intervals.

Further, as discussed above, the effective frequency may affect the duration required to obtain a therapeutically effective level of spinosyn in the feline's blood. By way of example, if the feline were being fed an spinosyn composition daily, the duration of feed administration required to achieve a therapeutically effective level of spinosyn in the feline's blood, and thus the “effective time,” would be comparatively less than if the feline were being fed the spinosyn composition only once or twice per week.

Further, the effective frequency is influenced by the amount of the daily dose in mg/kg of body weight of the feline. Particularly, at slightly higher daily doses, missed doses have less of an impact on efficacy.

Further, the effective frequency is influenced by the duration of treatment. In the initial stages, e.g., before the amount of spinosyn in the feline's blood has reached a therapeutically effective level, the animal feed may need to be administered more often than would be necessary after a longer period of use, i.e., once a therapeutically effective level is obtained.

For purposes of this disclosure, “substantially daily” means a sufficiently regular basis such that the spinosyn concentration in the feline's blood rises to and remains at a therapeutically effective level. For example, the disclosed feed composition can preferably be fed to a feline every day indefinitely. However, as a practical matter, there are many reasons why days may be missed or skipped periodically. For example, the feline may be ill or the owner may run out of the medicated feed composition. The disclosed method is robust enough that the feline will still be protected from fleas to some extent even with occasional interruptions in daily feeding of the medicated animal feed composition. In carrying out the method of this disclosure, the term “substantially daily” includes at least 10 days per month, more preferably at least 15 days per month, still more preferably at least 20 days per month. All of these feeding frequencies, whether they be, e.g., three times per week, every other day or daily, fit under the umbrella of substantially daily provided that they promote the spinosyn reaching and maintaining a therapeutically effective level of the spinosyn in the feline's blood.

The term “therapeutically effective” means that the dose or blood level of a spinosyn or a physiologically acceptable derivative thereof, or a metabolite thereof, is sufficient to control the flea infestation better than if no drug were present. The spinosyn or a physiologically acceptable derivative thereof, or a metabolite thereof, may be present on its own or with one or more additional active substances. Preferably it controls the flea infestation at around at least 50% better than if no drug were present, and more preferably it controls the flea infestation at about at least 90% better than if no drug were present.

In carrying out the methods of this disclosure, an effective or therapeutically effective amount of a spinosyn is administered orally to the feline. The term “effective amount” or “therapeutically effective amount” refers to the amount needed to control the flea infestation. As those skilled in the art will understand, this amount will vary depending upon a number of factors. These factors include, for example, the type of feline being treated and its weight and general physical condition.

In general, an effective amount refers to a dose of from about 0.18 to about 17 mg of the spinosyn/kg of body weight of the feline. More preferably, an effective amount refers to a dose of from about 0.3 to about 7.65 mg of the spinosyn/kg of body weight of the feline. More commonly, the effective amount is from about 0.3 to about 6.375 mg/kg of body weight of the feline.

Animal feeds will typically contain from about 0.0008 to about 0.34 percent of the spinosyn (by weight) in the feed; preferably between about 0.04 to about 0.2 percent of the spinosyn (by weight) in the feed; most preferably between about 0.0045 to about 0.1 percent of the spinosyn (by weight) in the feed.

While this disclosure describes concentrations of spinosyn in terms of feeds such as kibble, it also contemplates administration using other dosage forms, such as treats or chews. It is also contemplated that the spinosyn may be administered by itself or in a tablet, liquid, gel or other suitable form for oral administration. One of skill in the art will appreciate that the concentration of spinosyn will vary according to the particular dosage form. For example, where the animal feed is a treat, the concentration of spinosyn in the treat will be greater than, e.g., the concentration of spinosyn in a kibble. For example, if the daily dose of spinosyn based on the weight of the feline is 20 mg, then a typical 5 g treat may contain about 0.4 percent spinosyn (by weight). Since the amount of kibble consumed in a day is more than 5 g, the percent spinosyn in kibble will be smaller.

In one aspect, this disclosure relates to a method of controlling a flea infestation in a feline by administering a systemically active oral composition including a spinosyn, or a physiologically acceptable derivative or salt thereof, and animal feed or a chew at least once per week, more preferably three times per week, most preferably substantially daily.

In another aspect, this disclosure relates to a systemically active oral composition that includes a spinosyn and animal feed or a chew.

This disclosure also relates to the use of a spinosyn for the manufacture of an animal feed or a chew for controlling a flea infestation on a feline.

This disclosure also relates to a method of controlling a flea infestation on a feline for a prolonged time, comprising orally administering daily or substantially daily doses of an effective amount of a spinosyn to the feline. A daily feed is a feed that is intended to be administered daily, but which may be administered for effective times, as described herein. This method is especially useful for controlling fleas on a feline for a prolonged time comprising orally administering substantially daily doses of an effective amount of a spinosyn to the feline.

An aspect of this disclosure is the oral administration of an amount of spinosyn which is, in and of itself, ineffective or sub-optimal for controlling a flea infestation in a feline when administered in a single dose once per month, but over time with repeated administrations, as described herein, results in efficacious control of flea infestations. Ineffective or sub-optimal means that a single dosing, as well as several dosings, results in less than a 50% reduction in the flea infestation, including no, or substantially no, reduction, as compared to no drug administration at all. This reflects the chronic, rather than acute, administration aspect disclosed herein.

### EXAMPLES

The following examples illustrate the methods of this disclosure. It shall be understood that two of the following three examples are prophetic examples, as that term is understood to one of skill in the art. In other words, two of the three examples that follow are based on predicted results rather than actual experiments. However, the predicted results are based on experiments done with other animals and are believed to be sufficient for one of skill in the art to practice this disclosure.

### Example 1

Efficacy of Spinosyn Administered per os, i.e. by Mouth, to Cats for the Treatment and Control of Ctenocephalides felis.

Methods: A pool of 40 cats are to be preliminarily infested with ˜100 unfed adult C. felis in order to produce a cohort of cats that can suitably sustain a reliable infestation rate of approximately 50% of live fleas over a 48-hour period. The cats with the highest live flea counts are to be randomly allocated to 4 treatment groups (6 cats per group) based on their pre-treatment flea counts from experimental infestations. The first treatment group is to be the control group and groups 2-4 are to be the test groups.

The cats are to be housed individually during the study period and are to be fed a commercial dry cat food ration with ad libitum access to water.

Each cat in test groups 2-4 is to receive by mouth a liquid formulation of spinosyn, preferably spinosad. The dosage is to be administered to the cats on each of days 0-29 according to the test groups listed in TABLE 1.

TABLE 1

| Test Group | Oral Dose |
|---|---|
| 1 | 0 mg/kg (control group) |
| 2 | 0.425 mg/kg daily |
| 3 | 0.85 mg/kg daily |
| 4 | 1.7 mg/kg daily |

Cats in the control group are not to receive a spinosyn or any other flea control treatment. Each cat in test groups 2-4 is to be offered its daily ration (dry food) and the individual doses of liquid formulation are to be administered after the individual cat has eaten at least 25% of its total daily ration. After receiving the dose of a spinosyn the cats are to be allowed to continue eating. This mimics incorporating the spinosyn in feed. Each cat in test groups 2-4 and the control group is to be experimentally infested with 100 unfed adult fleas on test days −1, 5, 12, 19, 28 and 35. Comb counts for live adult fleas are to be conducted on days 2, 7, 14, 21, 30 and 37 with day 0 being the initiation of the daily dosing. The final experimental infestation is to occur approximately five days after the last daily dose of spinosyn.

The dosage level for test groups 2 and 3 would be expected to show greater than 90% efficacy in reducing fleas compared to the control group with 2 days of substantially daily dosing and greater than 98% efficacy after 1 week of substantially daily dosing. The dosage level for test group 4 would be expected to show greater than 98% efficacy in reducing fleas compared to the control group with 2 days of substantially daily dosing.

Further, all test groups would be expected to show greater than 85% efficacy a week after stopping the substantially daily dosing, e.g., at the 37-day comb count. At the higher dosage levels, e.g., test group 4, there may be no significant degradation in efficacy one week after stopping the substantially daily dosing.

Using the same study method as described above, blood is to be drawn at 72, 120, 168, 336, 504, 720 and 888 hours after the initial dose of spinosyn is administered. The average concentration of spinosyn in the blood for different dosage levels can then be determined.

The average plasma concentration of all spinosyns in a feline's blood with substantially daily dosing at the dosage level for test group 2 would be expected to range between about 20 ng/ml at 72 hours and about 58 ng/ml at 336 hours. For test group 3, the expected range would be between about 56 ng/ml at 72 hours and about 143 ng/ml at 504 hours. For test group 4, the expected range would be between about 118 ng/ml at 72 hours and about 372 ng/ml at 504 hours. By way of comparison, the plasma concentration of spinosad in a feline's blood using the typical single monthly dose would have an expected range between about 878 ng/ml one hour after the dose is given and 6107 ng/ml 12 hours after the dose is given.

### Example 2

Efficacy of Spinosyn in Cats when Administered in a Medicated Feed Dosed at 0.85 mg/kg of the Cat's Body Weight.

Methods: A pool of cats are to be preliminarily infested with ˜100 unfed adult C. felis in order to produce a cohort of 18 cats that can suitably sustain a reliable infestation rate of approximately 50% of live fleas over a 48-hour period. The cats with the highest live flea counts are to be randomly allocated to 3 groups (6 cats per group) based on their pre-treatment flea counts from experimental infestations. The first treatment group is to be the control group and groups 2-3 are to be the test groups.

The cats are to be housed individually during the study period and are to have ad libitum access to water.

There is to be an initial acclimation period of at least 4 days during which cats in test groups 2 and 3 are to be transitioned from a standard certified commercial cat feed to an unmedicated version of the daily feed. During the acclimation period, the cats are to be allowed 1 hour to consume the feed, after which the cats' acceptance of the feed will be observed and recorded.

Each cat in test groups 2 and 3 is to receive by mouth a daily feed formulation that includes a spinosyn, preferably spinosad. The dosage and formulation to be administered to the cats on each of days 0-29 according to test groups is shown in TABLE 2.

TABLE 2

| Test Group | Dose Contained in Daily Feed |
|---|---|
| 1 | 0 mg/kg (control group) |
| 2 | 0.85 mg/kg daily feed formulation 1 |
| 3 | 0.85 mg/kg daily feed formulation 2 |

Cats in the control group are not to receive a spinosyn or any other flea control treatment. On days 0-29, each cat in test groups 2 and 3 is to be offered its daily feed containing spinosyn for a period of 1 hour. On days 30-37 all cats will be given regular cat food, without spinosyn.

Each cat in test groups 2 and 3 and the control group is to be experimentally infested with 100 unfed adult fleas on test days −1, 5, 12, 28 and 35. Comb counts for live adult fleas are to be conducted on days 2, 7, 14, 30 and 37 with day 0 being the initiation of daily feeding of the medicated feed.

Assuming that all cats in groups 2 and 3 consume all of the food required for a daily 0.85 mg/kg dose without regurgitating and within the 1-hour time period, the percent reduction in live adult flea counts for test groups 2 and 3 would be expected to reach greater than 90% within 14 days. Some feed formulations may achieve 90% efficacy sooner than 14 days. Further, the percent reduction in live adult flea counts for test groups 2 and 3 would be expected to remain above 80% 7 days after the last administered dose. Differences in feed formulation would be expected to have less impact on the rate of decline in efficacy after dosing is stopped.

It can be appreciated by comparing the two examples that an effective amount of a spinosyn on average can be administered to a cat via medicated feed.

### Example 3

Efficacy of Spinosyn when Spinosyn is Administered in a Medicated Feed for the Treatment and Control of Ctenocephalides felis.

Methods: A pool of cats are to be preliminarily infested with ˜100 unfed adult C. felis fleas in order to produce cats that can suitably sustain a reliable infestation rate, defined as approximately 50% retention of live fleas at the end of a 48-hour period. The cats with the highest live flea counts are to be randomly assigned to a single control group (Group 1) and 2 medicated feed treatment groups (Groups 2 and 3) with 4 cats per group. The cats in groups 2 and 3 will receive spinosyn.

The cats are to be housed individually during the study period and are to have ad libitum access to water.

Each cat in a treatment group (Groups 2 and 3) is to receive a medicated daily feed from study days 0-29 according to TABLE 3.

TABLE 3

| | | Anticipated | Infestation |
|---|---|---|---|
| Treatment | # of | Daily Dose | with |
| Group | Cats | (mg/kg) | Fleas |
| 1 | 4 | 0 | Yes |
| 2 | 4 | 0.5 | Yes |
| 3 | 4 | 2.5 | Yes |

The cats are to be fasted overnight prior to each daily treatment. The daily dose volume of spinosyn is to be mixed into a small portion (approximately 25% of the cat's daily dietary needs) of wet canned cat food. Approximately 4 hours after the medicated feed has been initially offered, the remaining medicated feed, if any, will be mixed with the 75% unmedicated daily feed for each cat. The resulting feed offering may be consumed by the cats until the feed bowls are removed for the daily overnight fast.

Cats in the control group are not to receive spinosyn or any other flea control treatment. Each cat in treatment groups 2 and 3 and the control group is to be experimentally infested with 100 unfed adult C. felis on test days −1, 7, 14 and 28 during the treatment phase and on day 35 during the wash out period after the final feeding with the medicated daily feed. Comb counts for live and moribund fleas are to be conducted on days 2, 9, 16, 30 and 37.

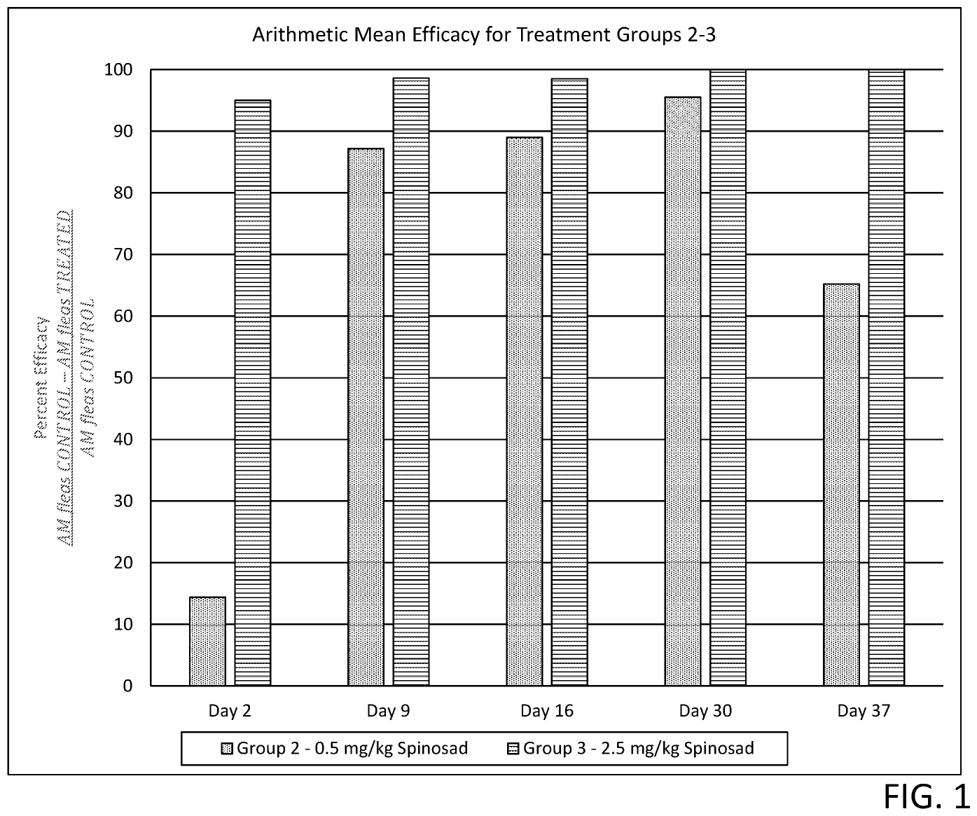

Results: Percent reduction in live and moribund flea counts for treatment groups 2 and 3 according to this example are shown in FIG. 1. As can be seen in FIG. 1, both treatment groups ultimately achieve over 90% efficacy.

While this invention has been described as having an exemplary design, the present invention may be further modified within the spirit and scope of this disclosure. This application is therefore intended to cover any variations, uses, or adaptations of the invention using its general principles.

What is claimed is:

1. A method of controlling a flea infestation in a feline in need thereof, comprising orally administering to said feline an effective amount of a spinosyn for an effective time at a frequency of at least 4 times per month.

2. The method of claim 1, wherein said feline is a domestic cat.

3. The method of claim 1, wherein said spinosyn is spinosad.

4. The method of claim 1, wherein said spinosyn is provided in a feed selected from the group consisting of a dry cat food and a wet cat food.

5. The method of claim 1, wherein said spinosyn is present in an amount of between about 7.5 mg/kg to 2400 mg/kg of a feed.

6. The method of claim 1, wherein said spinosyn is administered to said feline in an amount of between about 0.18 mg/kg and 17 mg/kg of body weight of said feline.

7. The method of claim 1, wherein the oral administration includes a feeding frequency selected from the group consisting of at least 3 times per week, substantially daily and daily.

8. The method of claim 1, wherein said effective time is selected from the group consisting of at least one week and at least two weeks.

9. The method of claim 1, wherein said administration provides a therapeutically effective level of spinosyn in said feline's blood within a time period selected from the group consisting of within one week of the first administration of said spinosyn and within two days of the first administration of said spinosyn.

10. The method of claim 1, wherein said administration provides a therapeutically effective level of spinosyn in said feline's blood for a period of time selected from the group consisting of: at least 45 days, at least 60 days, at least 90 days, at least 180 days and at least 365 days.

11. The method of claim 1, wherein said administration provides a concentration of spinosyn of between 15 ng/mL and 383 ng/mL in said feline's blood for a time period selected from the group consisting of at least 30 days, at least 60 days, at least 90 days, at least 180 days and at least 365 days.

12. The method of claim 1, wherein said spinosyn is administered for a number of days out of 30 days selected from the group consisting of at least 15 days and at least 20 days.

13. The method of claim 1, wherein said spinosyn is a component of a feed that comprises one or more other active substances.

14. The method of claim 1, further comprising discontinuing the administration of spinosyn for a number of days selected from the group consisting of at least 3 days and at least 7 days, wherein the feline's blood concentration of spinosyn is maintained at a therapeutically effective level.

15. The method of claim 14, further comprising resuming the administration of spinosyn after the discontinuing of the administration of spinosyn and thereby maintaining the feline's blood concentration of spinosyn at the therapeutically effective level.

16. The method of claim 1, wherein the oral administration includes a feeding frequency selected from the group consisting of at least 3 times per week, substantially daily and daily.

17. A feed or chew for controlling fleas in a feline, said feed to chew comprising a therapeutically effective among of a spinosyn to control a flea infestation when administered to said feline for an effective time at a frequency of at least four times per month.

18. The feed or chew according to claim 17, wherein the feline is a domestic cat.

19. The feed or chew according to claim 17, wherein said spinosyn is spinosad.

20. The feed or chew according to claim 17, wherein the spinosyn is in an amount of between about 7.5 mg/kg to 2400 mg/kg of a feed.
